SEQUENCE LISTING (2 sequences)

SEQ ID NO: 1 - DNA, 9060 bp, Zea mays
  misc_feature (15)...(2250): PTU #1
ccggaccgaagctttcagtgggggacaccgccggcagcagcgtaggggcatggagtcggctgctgcaggccgcacatcgccgtcagttgctgcgctatctgcgccgccaacagccccgcgacctggccgctttgcggctgctgctgcaggatggactggaggaccaagccgaagatcgcctggtaccggtgctgcggctccacctgcctgagctgctggcaacactgctgccgcaacgactggcactgaggcgagcagtagggcgtcgccgtcgggctgcactgatgcctcaggaactcgacgcactggcccaggatcggggtgctgccaacgccgcaggttccctgcagctggcacgggcttggatgcggctgttggcacgggcatgggtgtggctggggatgaggctgaggccccgggatccagctgcacgggtccaggaaagcaatcgcatagtcaagctaaatcatcaagatgcaaacttttcgcccttgctaaacacggtaaaattcgaatggacatgtgtggagcagcaaaggccttacgtccgagaaacagggccactcaacgagttagttaaattcaaagaaagaaacgcctccttgcaagttgcaacattcctagatcatactaatgaaaatgacgtccttcattaaagaacagggaagatagatttttgctccatagatcgtatgatgtgttcagccagactgtcggatggaccacacggtaatagcagtgctggacgatgttacatcgagaaagattactagccttttcatgggagtaaaggatataaaagaaataagttcaccacgattgcaggatagcatacaagatcagcgccactgcggcactgttcatcgaaaaaaaactgtggacgaagctttccccaaaattactcaacgaatcataaaccaagattagtcagatcaagagacagaggagaaacaaggcggacctttgcacgtcgacctgcaggcggccgcgaattcactagtgattcccggggcctcagcctcatccccagccacacccatgcccgtgccaacagccgcatccaagcccgtgccagctgcagggaacctgcggcgttggcagcaccccgatcctgggccagtgcgtcgagttcctgaggcatcagtgcagcccgacggcgacgccctactgctcgcctcagtgccagtcgttgcggcagcagtgttgccagcagctcaggcaggtggagccgcagcaccggtaccaggcgatcttcggcttggtcctccagtccatcctgcagcagcagccgcaaagcggccaggtcgcggggctgttggcggcgcagatagcgcagcaactgacggcgatgtgcggcctgcagcagccgactccatgcccctacgctgctgccggcggtgtcccccactgaaagcttggatccaatcgaattcccgcggccgccatggttggtacactattgtgctttctatgttgctagttggtctcaataatatgtgcgatatattagctactaatttaagagatatttatacacatctcagtccttgtgcttgtgcaaggttaattttggattttgcaaaaagaatattagtaagataaggtagagatgacatggtatctagttggttttgacatttagtgattactttgttgggttataatttatacgtaagcaagacaaacttgataaaatacaataaataagtggtttttcctctcggtattgtccacatacatgggtgacaatggtgtgatgcaatacctttacacatgtgtaacgatagccacatatgtgttgcttggtttggctattttccactagaaaaaaatcatggcacgattcaccattgttaattttagtgtggttaacttgtgtaataatcggtttaaagaatcactcgatccccaccgatagaaatttaggtacttttagtttcattttaatctataaagttgatactgattttttgatgccaacataaaggcgcctatgtgatttaaacataatgaatatgagcaaaataaatagtaacatataaggaagacattgctagttgatatagtcgtactggttgtaatagtgcagtgttgttgctatgtcgactcaatcacaacaaaaaacactaggaagaagcggaggaagtaaggagttgtgtcgctgatatttccctagaacacgattgtcgtccttcatctgagtagatattcatttcggaggcctgctcagtgctgtaatggagtaccagcccgggtaccgagctcgaattccggtccgggtaacgtcacccggtccgggcctagaaggccatttaaatcctgaggatctggtcttcctaaggacccgggatatcgctatcaactttgtatagaaaagttgggccgaattcgagctcggtacggccagaatggcccggaccgggttaccgaattcgagctcggtaccctgggatccgatatcgatgggccctggccgaagctttagccatggcgacggcgatgggcgcgatggccgccacctgcatggcctcgatcccggccgccgctacgttccccggtgatctcggcgtcggacgacgccgagcggttgtaccagggtggcgcgcgcgccggtgggagacggctgcgtgcgacgccgcctgcccggaggccgttcctgttctcgccgaagggcgtttcggactcgcggagctcgcaaacgtgccttgatccggacgccagcacgagtgttcttgggatcatccttggagggggtgctgggacaaggctgtatccactgacgaagaagagggcgaaaccagcagtgccgttgggcgccaactacaggctcatagatctagacccggtggcgccgcgatgcagcggtcgccggtggagaagcctccgttcactctgggtcagatcaagaaggccatcccgccacactgcttcgagcgctcggtgctcaagtccttctcgtacgtggtccacgacctggtgatcgccgcggcgctcctctacttcgcgctggccatcataccggcgctcccaagcccgctccgctacgccgcctggccgctgtactggatcgcgcaggggtgcgtgtgcaccggcgtgtgggtcatcgcgcacgagtgcggccaccacgccttctcggactagccgctgtacctggcgaccaacgcgtcggggcggccgtacccgcgcttcgcctgccacttcgacccctacggccccatctacaacgaccgggagcgcgcccagatcttcgtctcggacgccggcgtcgtggccgtggcgttcgggctgtaccccgggatccagctgcacgggtccaggaaagcaatcgcatagtcaagctaaatcatcaagatgcaaacttttcgcccttgctaaacacggtaaaattcgaatggacatgtgtggagcagcaaaggccttacgtccgagaaacagggccactcaacgagttagttaaattcaaagaaagaaacgcctccttgcaagttgcaacattcctagatcatactaatgaaaatgacgtccttcattaaagaacagggaagatagatttttgctccatagatcgtatgatgtgttcagccagactgtcggatggaccacacggtaatagcagtgctggacgatgttacatcgagaaagattactagccttttcatgggagtaaaggatataaaagaaataagttcaccacgattgcaggatagcatacaagatcagcgccactgcggcactgttcatcgaaaaaaaactgtggacgaagctttccccaaaattactcaacgaatcataaaccaagattagtcagatcaagagacagaggagaaacaaggcggacctttgcacgtcgacccggggtacagcccgaacgccacggccacgacgccggcgtccgagacgaagatctgggcgcgctcccggtcgttgtagatggggccgtaggggtcgaagtggcaggcgaagcgcgggtacggccgccccgacgcgttggtcgccaggtacagcggctagtccgagaaggcgtggtggccgcactcgtgcgcgatgacccacacgccggtgcacacgcacccctgcgcgatccagtacagcggccaggcggcgtagcggagcgggcttgggagcgccggtatgatggccagcgcgaagtagaggagcgccgcggcgatcaccaggtcgtggaccacgtacgagaaggacttgagcaccgagcgctcgaagcagtgtggcgggatggccttcttgatctgacccagagtgaacggaggcttctccaccggcgaccgctgcatcgcggcgccaccgggtctagatctatgagcctgtagttggcgcccaacggcactgctggtttcgccctcttcttcgtcagtggatacagccttgtcccagcaccccctccaaggatgatcccaagaacactcgtgctggcgtccggatcaaggcacgtttgcgagctccgcgagtccgaaacgcccttcggcgagaacaggaacggcctccgggcaggcggcgtcgcacgcagccgtctcccaccggcgcgcgcgccaccctggtacaaccgctcggcgtcgtccgacgccgagatcaccggggaacgtagcggcggccgggatcgaggccatgcaggtggcggccatcgcgcccatcgccgtcgccatggatccgatatcttaaggtaggctagcagagcgagctaggtaccccacgtgcgcacgctgcccagagctcctgccgctgccgctgccgctgatgcttgagctacgactacgagtgaggtagatgagggcgagggagagctgggtgtttatagagggcgtggcaacgcgcggaggcgggatctggcgcggccgcgtgggacgcatgcgggactcgtgtcgggggcagcgcgacacctgtgtagggggtgtcgaggacaaggcttcctcggtcgcgccgcttccgcgcggcgatatatacgaggttgtgtgtgtgcgcgcactgccgttggatcggccgctggttgccggaggtggcggggccgaatctcatgtgggccgtagtccgggcctcttttacttcttttgtcttccgtgtctcactattttctggtccacgtagacctacatcacatactagcaagaactagtaaaagcatctgagcgtcagtatagttttagtatattaataataatatagatttattgtgactaaaatataatttgtggaaacaatgtgcttggtacctgttgagattttgtgaagaactacagcatgacaacatatttaaattgaggacacttcctctcttctccgaagaggatgaatggtagggtgtccagaaaacaaatttgtatatcgaaactcatgagaagttacgaagcgtaaaatatggattatcgtaattatttgctgaaggtgagatgcatgttctctcaatcgattaaccgtgtaatgctcattgccaataataatatcctagggaaagactcgatatatgatgaaagacaagacaacaataatcttcgccatcttatacctttggaggttctctaaacagggggtacacgggcgtagggatacaggaatgccaatcccgatagcagatatatttgaggtgtttggtttgaggaatgagatagtcaatcggatcgtaccctgggatccgatatcgatgggccctggccgaagcttggtcacccggtccgggcctagaaggccagcttcaagtttgtacaaaaaagcaggctccggccagaatggcccggaccgggttacccggaccggaattcgattaagcttgatatcgcagcggggccgatgaagtcgaagcagcagggtcgacgacaagatcgcgagcagtcgcgtgatgacgcttactaaaaaccttattcgccctctcccgatgcaagatctcgaatgcgaagggttccggagacctgctctcctgaacgcagatgcacgtcggcgattgggatggcggcgcagttgtgagcagagggccttatatagggccgtgtccgcgaaccgataacgatccgcgatccgatctacatgttatctacgaatcgatagactcgcgttccgttcagatccttatcgcgatcggttagggctctaacttaacagccaagcaaccgccctggcccggcgaggcgagcgcgtgtggttctccacactctctcctctcatccatgacttgttgagtgagtgtagcatccatatttaaactagttccactccacttggactagcaatatggcactatatgttccaccattccctagccataccatacatgggtttttgagatttttttagaatttaattaaatttctcaattgagtctagcccataaatccaacacgatataagtctatctgtcgctggtagattgagagaggatgtgtgcatgtctgtaaataaaaaaaagttgtttttacacataaattgcgctatgactttacatgaaataaattttctaaaatttaaaacttacataagtaaaaaaaaatataaagaaggaaagaaacacgacatggaaaaaatctctcgttgttttatatggatgcaacagctgcagtccccgtgcaagcgatgctcatccgttcccatggcgtgcacggcccagaaacgacacgcttcacctactttcttccctgccaccacacccaccgtccacccacaccacaccgcgcgccacgcgcccacggcacctcggctcagtgtcgtcgcatgtcgctcacgtactgtcgcagaactcacaccgtcacacggtgcctgctatctagctaatgctgctagcagccatgtcacaccgatataacccggccaccgcgcgccgcgccacgtcgccatgcacgcggccacgtcccccatcgatcgacgtcgtcctcctcatcctggctcctccggatccatccccgcgctcctataaatacctcgtctcatgctcggccatgtacatcgacccagccatctcctcaccctcgttcaccacacagtccgccactcctttagtagcttgtgatttgtacgtcgacgagatcactggtcgggtgagccatggagagatctcaacggcagtctcctccgccaccgtcgccgtcctcctcctcgtcctccgtctccgcggacaccgtcctcgtccctcccggaaagaggcggagggcggcgacggccaaggccggcgccgagcctaataagaggatccgcaaggaccccgccgccgccgccgcggggaagaggagctccgtctacaggggagtcaccaggcacaggtggacgggcaggttcgaggcgcatctctgggacaagcactgcctcgccgcgctccacaacaagaagaaaggcaggcaagtctacctgggggcgtatgacagcgaggaggcagctgctcgtgcctatgacctcgcagctctcaagtactggggtcctgagactctgctcaacttccctgtggaggattactccagcgagatgccggagatggaggccgtgtcccgggaggagtacctggcctccctccgccgcaggagcagcggcttctccaggggcgtctccaagtacagaggcgtcgccaggcatcaccacaacgggaggtgggaggcacggattgggcgagtctttgggaacaagtacctctacttgggaacatttgacactcaagaagaggcagccaaggcctatgaccttgcggccattgaataccgtggcgtcaatgctgtaaccaacttcgacatcagctgctacctggaccacccgctgttcctggcacagctccaacaggagccacaggtggtgccggcactcaaccaagaacctcaacctgatcagagcgaaaccggaactacagagcaagagccggagtcaagcgaagccaagacaccggatggcagtgcagaacccgatgagaacgcggtgcctgacgacaccgcggagcccctcaccacagtcgacgacagcatcgaagagggcttgtggagcccttgcatggattacgagctagacaccatgtcgagaccaaactttggcagctcaatcaatctgagcgagtggttcgctgacgcagacttcgactgcaacatcggatgcctgttcgatgggtgttctgcggctgacgaaggaagcaaggatggtgtaggtctggcagatttcagtctgtttgaggcaggtgatgtccagctgaaggatgttctttcggatatggaagaggggatacaacctccagcgatgatcagtgtgtgcaactaatctagagatcaatcactagacccgggtaataaaagggtagtgtacgcctaccgcgtacgtacgtgtcaccgggcgtggcactctccagtctccagggacccatccaccaaatgctactgctccttcgtagggagacgtgggaataaagagtggtagctgcatgcacgtacggcggccatggctctccgatgagagagctagctgtgtacgtgtgttcttgatgttgttccatgcatgacatgtatacgtcttgcctaagtacgcttgtactagttgagagactgtgtaagtgaaatgtgctataataataaataagtaaaaggcgccttctccaacattctatgggctcgtttgggagggttgcggctcctctcaaaacggctccagctccaactcctccaaaggagtagctcctctgcaggagcccatgctttttgcaaatcgtttgctaaaaaacggctccttgtgtatgccgtgctcatcatcatcaacgttgattacctagaaaccactgcagtgctttctgttgtcggatagtggaaggctccttgtgtattattacaacaaaaaaatattatggagtaatattagaaaagcatttgcacatcacaatccatacacaagtcatatatcacttggatgattacctagaaagaaagatcgctcttgcgcgatgtcatcacgaaacctatccatcatacgatcattagagtatggacatgatgagtcagttgtatttctatatctaaaaggtatagtgggcacgtaatctggatttcgatcgcacttatagggcccaatcgaattcccgcggccgccatggcggcc

SEQ ID NO: 2 - DNA, 8040 bp, Zea mays
  misc_feature (14)...(2586): PTU #1
aagcaagcggcggctagaacttgctgttgtaccagaagacgcccttgcggttgcggttctcaggctcgacgtagatgcactccctggcctcgcgccaggtggccttggcgacaggggtcgggtcgaactggtagtactcgccgaggacgggcctgatcgccctggtggcctccacggcgtggtagtgcggcatggtggagaagaggtggtgcgcgacgtgcgtgtccgtgatgtggtggaacacgcggttgaggacgccgtagtctcggtcgacggtggcgagcgcgccgcgcagccagtcccactcgcccgagtcgtagtggggcagcgccgggtgggtgtgctgcaggtacgtgatgagcaccagccacgcgttgacgatcagcagcggcacggcgtacacgcgcaccacccaccagagcccgaacgccgccgccagcttgtacagcccgaacgccacggccgcgacgccggcgtccgagacgaagatctgggcgcgctcccggtcgccgtagatcgggccgtaggggtcgaagtggcaggcgaagcgcgggtacgggcgccccgacgcgttggtgggctccgtgctgaggtccttctcgtacgtggcccacgacctgtcgctcgccgcggcgctcctctacctcgcggtggccgtgataccggcgctaccctgcccgctccgctacgcggcctggccgctgtactgggtggcccaggggtgcgtgtgcacgggcgtgtgggtgatcgcgcacgagtgcggccaccacgccttctccgaccacgcgctcctggacgacgccgtcggcctggcgctgcactcggcgctgctggtgccctacttctcgtggaagtacagccaccggcgccaccactccaacacggggtccctggagcgcgacgaggtgttcgtgccgaggaccaaggaggcgctgccgtggtacgccccgtacgtgcacggcagccccgcgggccggctggcgcacgtcgccgtgcagctcaccctgggctggccgctgtacctggccaccaacgcgtcggggcgcccgtacccgcgcttcgcctgccacttcgacccctacggcccgatctacggcgaccgggagcgcgcccagatcttcgtctcggacgccggcgtcgcggccgtggcgttcgggctgtacaagctggcggcggcgttcgggctctggtgggtggtgcgcgtgtacgccgtgccgctgctgatcgtcaacgcgtggctggtgctcatcacgtacctgcagcacacccacccggcgctgccccactacgactcgggcgagtgggactggctgcgcggcgcgctcgccaccgtcgaccgagactacggcgtcctcaaccgcgtgttccaccacatcacggacacgcacgtcgcgcaccacctcttctccaccatgccgcactaccacgccgtggaggccaccagggcgatcaggcccgtcctcggcgagtactaccagttcgacccgacccctgtcgccaaggccacctggcgcgaggccagggagtgcatctacgtcgagcctgagaaccgcaaccgcaagggcgtcttctggtacaacagcaagttctagccgccgcttgctttttccctagaggatccgatatcttaaggtaggctagcagagcgagctaggtaccccacgtgcgcacgctgcccagagctcctgccgctgccgctgccgctgatgcttgagctacgactacgagtgaggtagatgagggcgagggagagctgggtgtttatagagggcgtggcaacgcgcggaggcgggatctggcgcggccgcgtgggacgcatgcgggactcgtgtcgggggcagcgcgacacctgtgtagggggtgtcgaggacaaggcttcctcggtcgcgccgcttccgcgcggcgatatatacgaggttgtgtgtgtgcgcgcactgccgttggatcggccgctggttgccggaggtggcggggccgaatctcatgtgggccgtagtccgggcctcttttacttcttttgtcttccgtgtctcactattttctggtccacgtagacctacatcacatactagcaagaactagtaaaagcatctgagcgtcagtatagttttagtatattaataataatatagatttattgtgactaaaatataatttgtggaaacaatgtgcttggtacctgttgagattttgtgaagaactacagcatgacaacatatttaaattgaggacacttcctctcttctccgaagaggatgaatggtagggtgtccagaaaacaaatttgtatatcgaaactcatgagaagttacgaagcgtaaaatatggattatcgtaattatttgctgaaggtgagatgcatgttctctcaatcgattaaccgtgtaatgctcattgccaataataatatcctagggaaagactcgatatatgatgaaagacaagacaacaataatcttcgccatcttatacctttggaggttctctaaacagggggtacacgggcgtagggatacaggaatgccaatcccgatagcagatatatttgaggtgtttggtttgaggaatgagatagtcaatcggatcgtaccctgggatccgatatcgatgggccctggccgaagcttggtcacccggtccgggcctagaaggccagcttcaagtttgtacaaaaaagcaggctccggccagaatccggtaagtgactagggtcacgtgaccctagtcacttaaattcggccagaatggcccggaccgggttacgtgacccggaccgaagctttcagtgggggacaccgccggcagcagcgtaggggcatggagtcggctgctgcaggccgcacatcgccgtcagttgctgcgctatctgcgccgccaacagccccgcgacctggccgctttgcggctgctgctgcaggatggactggaggaccaagccgaagatcgcctggtaccggtgctgcggctccacctgcctgagctgctggcaacactgctgccgcaacgactggcactgaggcgagcagtagggcgtcgccgtcgggctgcactgatgcctcaggaactcgacgcactggcccaggatcggggtgctgccaacgccgcaggttccctgcagctggcacgggcttggatgcggctgttggcacgggcatgggtgtggctggggatgaggctgaggccccgggatccagctgcacgggtccaggaaagcaatcgcatagtcaagctaaatcatcaagatgcaaacttttcgcccttgctaaacacggtaaaattcgaatggacatgtgtggagcagcaaaggccttacgtccgagaaacagggccactcaacgagttagttaaattcaaagaaagaaacgcctccttgcaagttgcaacattcctagatcatactaatgaaaatgacgtccttcattaaagaacagggaagatagatttttgctccatagatcgtatgatgtgttcagccagactgtcggatggaccacacggtaatagcagtgctggacgatgttacatcgagaaagattactagccttttcatgggagtaaaggatataaaagaaataagttcaccacgattgcaggatagcatacaagatcagcgccactgcggcactgttcatcgaaaaaaaactgtggacgaagctttccccaaaattactcaacgaatcataaaccaagattagtcagatcaagagacagaggagaaacaaggcggacctttgcacgtcgacctgcaggcggccgcgaattcactagtgattcccggggcctcagcctcatccccagccacacccatgcccgtgccaacagccgcatccaagcccgtgccagctgcagggaacctgcggcgttggcagcaccccgatcctgggccagtgcgtcgagttcctgaggcatcagtgcagcccgacggcgacgccctactgctcgcctcagtgccagtcgttgcggcagcagtgttgccagcagctcaggcaggtggagccgcagcaccggtaccaggcgatcttcggcttggtcctccagtccatcctgcagcagcagccgcaaagcggccaggtcgcggggctgttggcggcgcagatagcgcagcaactgacggcgatgtgcggcctgcagcagccgactccatgcccctacgctgctgccggcggtgtcccccactgaaagcttggatccaatgaattcccgcccatggttggtacactattgtgctttctatgttgctagttggtctcaataatatgtgcgatatattagctactaatttaagagatatttatacacatctcagtccttgtgcttgtgcaaggttaattttggattttgcaaaaagaatattagtaagataaggtagagatgacatggtatctagttggttttgacatttagtgattactttgttgggttataatttatacgtaagcaagacaaacttgataaaatacaataaataagtggtttttcctctcggtattgtccacatacatgggtgacaatggtgtgatgcaatacctttacacatgtgtaacgatagccacatatgtgttgcttggtttggctattttccactagaaaaaaatcatggcacgattcaccattgttaattttagtgtggttaacttgtgtaataatcggtttaaagaatcactcgatccccaccgatagaaatttaggtacttttagtttcattttaatctataaagttgatactgattttttgatgccaacataaaggcgcctatgtgatttaaacataatgaatatgagcaaaataaatagtaacatataaggaagacattgctagttgatatagtcgtactggttgtaatagtgcagtgttgttgctatgtcgactcaatcacaacaaaaaacactaggaagaagcggaggaagtaaggagttgtgtcgctgatatttccctagaacacgattgtcgtccttcatctgagtagatattcatttcggaggcctgctcagtgctgtaatggagtaccagcccgggtaccgagctcgaattccggtccgggtaacgtcacccggtccgggcctagaaggcccggaccgattaaactttaattcggtccgggttaccagagctggtcacctaagtgactagggtcacgtgaccctagtcacttattcccgggcacccagctttcttgtacaaagtggccgttaacggatcggccagaatggcccggaccgggttagatccgattgactatctcattcctcaaaccaaacacctcaaatatatctgctatcgggattggcattcctgtatccctacgcccgtgtaccccctgtttagagaacctccaaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacaaaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggcagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaagatatctgcagaattcgcccttcgcatggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatagaagggcgaattccagcacactggcggccgttactagtggatctgtcgactctagacccgggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgt

(19) United States
(12) Patent Application Publication
(10) Pub. No.: US 2011/0126319 A1
(43) Pub. Date: May 26, 2011

(54) Plants Having Increased Oil, Oleic Acid Content and Digestibility and Methods of Producing Same

(75) Inventors: Sally A. Catron, Altoona, IA (US); Chun-Hsiang Chang, West Des Moines, IA (US); Kimberly F. Glassman, Ankeny, IA (US); Todd Leister, Ankeny, IA (US); Mitchell C. Tarczynski, Johnston, IA (US)

(73) Assignee: PIONEER HI-BRED INTERNATIONAL, INC., Johnston, IA (US)

(21) Appl. No.: 12/945,530

(22) Filed: Nov. 12, 2010

Related U.S. Application Data
(60) Provisional application No. 61/263,862, filed on Nov. 24, 2009.

(51) Int. Cl. A01H 1/00; A01H 5/00; C07H 21/04

(52) U.S. Cl. 800/281; 800/320.1; 536/23.1

(57) ABSTRACT

The present invention is directed to compositions and methods for producing corn plants and grain having increased oil content, increased oleic acid content of the oil, and increased digestibility over commodity corn grain. The resulting grain finds use in agricultural and industrial applications.

### CROSS REFERENCE TO RELATED APPLICATION

This application claims the benefit of U.S. Application Ser. No. 61/263,862 filed Nov. 24, 2009, herein incorporated by reference.

### FIELD OF THE INVENTION

The invention relates to the field of plant molecular biology and the use of genetic modification to improve the quality of crop plants, more particularly to methods for improving the nutritional value of grain for animal feed and industrial processes.

### BACKGROUND OF THE INVENTION

Corn is a versatile crop used in a wide variety of applications. It can be used as a human food source, an animal feed, a source of energy in such processes as ethanol production, and is a source of carbohydrate, oil, protein and fiber. Seed produced from corn is the source of much of its value, and can be alternately referred to as a kernel, or grain. Corn grain consists of a seed coat, the outer layer, which can also be referred to as the pericarp, bran or fiber. The endosperm of the grain is a source of starch and contains a majority of the zein protein fraction. The embryo, which can also be called the germ, is the primary source of oil or lipids from the plant.

Most corn grain is handled as a commodity, since many of the industrial and animal feed requirements for corn can be met by common varieties of field corn which are widely grown and produced in volume. However, there exists at present a growing market for corn with special end-use properties which are not met by corn grain of standard composition.

Oil content in corn which has not otherwise been modified for increased oil content (i.e.: commodity corn, such as the standard number 2 yellow dent), is about 4.4%, on a dry weight basis and can range from 2.5% to 5.1% of the whole kernel. Grain with increased oil content provide additional value from each plant produced when used as the source of oil for human or animal consumption, or in industrial applications. Efforts to increase oil content of corn, have resulted in hybrids with more than 6% dry weight oil content but are lower in yield than hybrids with lower levels of oil. Also, corn bred to contain higher oil generally achieves this through an increase in embryo size which negatively impacts milling processes. Corn grain with increased oil but normal embryo size is needed.

Increased amounts of unsaturated fatty acids in livestock feed creates value for both the livestock producer and food processor. The double bonds of poly-unsaturated fatty acids are susceptible to oxidation by free radicals which reduces meat quality over time. In contrast, feeding mono-saturated fatty acids such as oleic acid which are less prone to oxidation, increases meat shelf life. Corn which is not modified to have increase oleic acid content typically has less than 30% oleic acid content on a dry weight basis in the oil extracted from corn kernels.

Another important measure of grain quality when used as an animal feed is digestibility: that is, the amount of the grain the animal can digest and use for energy and its impact on the animal itself. An increase in digestibility increases the nutritional value of feed and helps to reduce feeding costs to the livestock producer. Commodity corn grain has a digestibility percentage of about 86% to 87% on average.

Breeding efforts to bring desired increased levels of all three characteristics of oil, oleic acid, and digestibility have to date been unsuccessful. Thus there is a need in the art for corn plants having these characteristics and methods for producing them.

### DETAILED DESCRIPTION

The present invention is directed to compositions and methods for producing corn plants and grain having increased oil content, increased oleic acid content of the oil, and increased digestibility.

The invention is directed to corn grain having an oil content that is at least about 15% higher on a dry weight basis without an increase in embryo size and digestibility of at least about 0.5% higher than a null plant, and also has an oleic acid content of at least about 60% on a weight basis of the grain oil. Another embodiment provides oil content at least about 20%, at least about 25%, at least about 30% higher or any percentage in-between. A further embodiment provides for oleic acid content of the oil that is at least about 65%, at least about 70%, 80% or 85% or higher or any percentage in-between. Another embodiment provides digestibility is at least about 2% higher than grain not modified for increased digestibility.

Typically, “grain” means the mature kernel produced by commercial growers for purposes other than growing or reproducing the species, and “seed” means the mature kernel used for growing or reproducing the species. For the purposes of the present invention, “grain”, “seed”, and “kernel”, will be used interchangeably.

A method of producing such grain is provided by introducing into a plant cell a plant transcription unit comprising a promoter which expresses in the embryo operably linked to an ODP1 gene operably linked to plant transcription unit comprising a promoter which expresses in the embryo operably linked to two complementary fragments of an FAD2-1 gene, and a plant transcription unit comprising a promoter which expresses in the endosperm operably linked to two complementary fragments of a 27 kDa gamma zein gene.

Still another method provided for producing such grain is introducing into a plant cell a plant transcription unit comprising a promoter which expresses in the embryo operably linked to a ZmDGAT1-2(ASK) gene, a plant transcription unit comprising a promoter which expresses in the embryo operably linked to two complementary fragments of an FAD2-2 gene, and a plant transcription unit comprising a promoter which expresses in the endosperm operably linked to two complementary fragments of a 27 kDa gamma zein gene.

Oil content of corn grain not modified to have high oil or high oleic content has an oil content ranging from 2.5% to 5.1% oil on a dry weight basis and an oleic acid content of about 25% to 30% on a weight basis of the oil. See Bergquist et al, U.S. Pat. No. 5,706,603. Digestibility of unmodified corn grain is averaged at 86% to 87% based on lab analysis (see Example 6). Yellow dent number 2 corn is an example of such an unmodified plant.

The invention provides for a corn plant and grain produced wherefrom in which the grain has increased oil content without increasing embryo size, increased oleic acid content and increased digestibility compared to null grain. When referring to a null plant, cell or grain, is meant a plant, plant cell, or grain of same: 1) which has the same genotypic background but has not been transformed with a construct that has a known effect on the trait of interest; 2) a wild-type plant, cell, or grain of the same genotype as the starting material for the genetic alternation which results in the subject plant, cell, or grain; 3) a plant, cell, or grain which is a non-transformed segregant among progeny of a subject plant, cell, or grain; 4) a plant, cell, or grain genetically identical to the subject plant, cell, or grain but not exposed to conditions or stimuli which would induce expression of the genes in the constructs described herein; or 5) the subject plant, cell, or grain under conditions in which such genes are not expressed.

The increased oil content of the grain of the invention is at least about 15%, 20%, 25% or 30% or higher or any range in-between, on a dry weight basis compared to null grain. The increase can also be on about a 0.5-fold, 1-fold, 1.5-fold, 2-fold, 2.5-fold, 3-fold, basis or more when compared to null grain. Increased oil content includes any increase in the level of oil in the grain.

The increased digestibility of the grain of the invention is at least about 0.5%, at least about 1%, at least about 1.5%, at least about 2%, at least about 2.5%, at least about 3% or more higher, or any range in-between, when compared to null grain. The increase can also be on a 0.5-fold, 1-fold, 1.5-fold, 2-fold or more basis when compared to null grain.

The increased oleic acid content of grain of the invention is at least about 60%, at least about 65%, at least about 70%, at least about 75%, at least about 80%, at least about 85% or more, or any range in-between, of the oleic content on a weight basis.

Oil and/or oil constituents, such as oleic acid and linoleic acid, can be measured by any method known in the art. Methods of measuring oil and oil constituents in maize kernels, dissected germ, and endosperm are disclosed, for example, in Ravenello et al., WO 2005/003312 and U.S. Pat. No. 7,179,956, and Thompson et al., WO 02/062129.

Means for measuring digestibility are also well known to those skilled in the art. “Digestibility” is the fraction of the feed or food that is not excreted in feces or urine. Digestibility is a component of energy availability. It can be further defined as digestibility of specific constituents (such as carbohydrates or protein) by determining the concentration of these constituents in the foodstuff and in the excreta.

Digestibility can be estimated using in vitro assays, which is routinely done to screen large numbers of different food ingredients and plant varieties. In vitro techniques, including assays with rumen inocula and/or enzymes for ruminant livestock (e.g. Pell and Schofield, Journal of Dairy Science 76(4):1063-1073 (1993)) and various combinations of enzymes for monogastric animals reviewed in Boisen and Eggum, Nutrition Research Reviews 4:141-162 (1991) are also useful techniques for screening transgenic materials for which only limited sample is available.

The enzyme digestible dry matter (EDDM) assay used in the present invention as an indicator of in vivo digestibility is known in the art and can be performed according to the methods described in Boisen and Fernandez (1997) Animal Feed Science and Technology 68:277-286, and Boisen and Fernandez (1995) Animal Feed Science and Technology 51:29-43. The specifics of any assay can vary and the in vitro method used for determining EDDM described in the examples below is a modified version of the above protocol.

As used herein, “genetically modified” or “genetically altered” means the modified expression of a seed protein resulting from one or more genetic modifications; the modifications including but not limited to: recombinant gene technologies, induced mutations, and breeding stably genetically modified plants to produce progeny comprising the altered gene product.

In an embodiment of the invention, the grain of the invention is produced by introducing into a plant or plant cell a combination of nucleic acid molecules. When referring to “introduction” of the nucleic acid molecules into a plant, it is meant that this can occur by direct transformation methods, such as Agrobacterium transformation of plant tissue, microprojectile bombardment, electroporation, or any one of many methods known to one skilled in the art; or, it can occur by breeding a plant having the heterologous nucleotide sequence with another plant so that the progeny have the nucleic acid molecules incorporated into their genomes. Such breeding techniques are well known to those of skill in the art.

For a discussion of plant breeding techniques, see Poehlman (1995) Breeding Field Crops. AVI Publication Co., Westport Conn., 4^{th} Edit. Backcrossing methods may be used to introduce a gene into the plants. A description of this and other plant breeding methodologies can be found in references such as Poehlman, supra, and Plant Breeding Methodology, edit. Neal Jensen, John Wiley & Sons, Inc. (1988).

In one embodiment of the invention, the method comprises introducing into the plant cell plant transcription units (PTU) comprising various genetic material. The term “plant transcription unit” is meant to refer to operably linked genetic components that are transferred into the plant cell such that the components can function with one another. It is not intended to imply that a separate vector must be employed for each PTU. Rather, the genetic material of the PTUs may be introduced in any manner convenient (and as further described below) such that the material functions in the cell to increase the oil and oleic content and digestibility of the resulting grain as described herein. Plant transcription units may also be referred to as “cassettes” generally in the context of vector construction.

The invention provides for plant transcription units that interfere with expression of the targeted gene. The nucleic acid sequences for use in the methods of the invention can be provided as co-suppression units for transcription in the plant of interest.

For the purpose of this invention the term “co-suppression” is used to collectively designate gene silencing methods based on mechanisms involving the expression of sense RNA molecules, aberrant RNA molecules, double-stranded RNA molecules, micro RNA molecules and the like. Transcription units can contain coding and/or non-coding regions of the genes of interest. Additionally, transcription units can contain promoter sequences with or without coding or non-coding regions. The transcription units may include 5′ (but not necessarily 3′) regulatory sequences, operably linked to at least one of the sequences of the invention.

Methods of co-suppression are known in the art and can be similarly applied to the instant invention. These methods involve the silencing of a targeted gene by spliced hairpin RNA's and similar methods also called RNA interference (RNAi) and promoter silencing (see Smith et al. (2000) Nature 407:319-320, Waterhouse and Helliwell (2003)) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Plant Physiol. 129:1723-1731; and Patent Application WO 99/53050; WO 99/49029; WO 99/61631; WO 00/49035 and U.S. Pat. No. 6,506,559).

In one embodiment, co-suppression transcription units can comprise sequences of the invention in so-called “inverted repeat” structures. The transcription units may additionally contain a second copy of the fragment in opposite direction to form an inverted repeat structure: opposing arms of the structure may or may not be interrupted by any nucleotide sequence related or unrelated to the nucleotide sequences of the invention. (see Fiers et al. U.S. Pat. No. 6,506,559). The transcriptional units are linked to be co-transformed into the organism. Alternatively, additional components can be provided in multiple over-expression and co-suppression transcriptional units.

In another embodiment, co-suppression transcription units can comprise a promoter that drives transcription in the plant operably linked to at least one nucleic acid sequence in the sense orientation encoding at least a portion of the protein of interest.

Methods for suppressing gene expression in plants using nucleotide sequences in the sense orientation are known in the art. The methods generally involve transforming plants with a DNA construct comprising a promoter that drives transcription in a plant operably linked to at least a portion of a nucleotide sequence that corresponds to the transcript of the endogenous gene. Typically, such a nucleotide sequence has substantial sequence identity to the sequence of the transcript of the endogenous gene, at least about 65%, 70%, 75%, 80%, 85%, 90%, 91%, 92%, 93%, 94%, 95%, 96%, 97%, 98%, or 99% or more sequence identity over the entire length of the sequence. Furthermore, portions, rather than the entire nucleotide sequence, of the polynucleotides may be used to disrupt the expression of the target gene product. Generally, sequences of at least 10, 15, 20, 21, 22, 23, 24, 25, 26, 27, 28, 29, 30, 35, 40, 45, 50, 55, 60, 65, 70, 75, 80, 85, 90, 95, 100, 200 nucleotides, or greater may be used. See U.S. Pat. Nos. 5,283,184 and 5,034,323; herein incorporated by reference.

A plant transcription unit for co-suppression may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, International Publication No. WO 02/00904.

In other embodiments, inhibition of the expression of a protein of interest may be obtained by RNA interference by expression of a gene encoding a micro RNA (miRNA). miRNAs are regulatory agents consisting of about 22 ribonucleotides. Micro RNA are highly efficient at inhibiting the expression of endogenous genes and the RNA interference they induce is inherited by subsequent generations of plants. See, for example Javier et al. (2003) Nature 425: 257-263.

For miRNA inhibition, the plant transcription unit is designed to express an RNA molecule that is modeled on an endogenous miRNA gene. The miRNA gene encodes an RNA that forms a hairpin structure containing a 22-nucleotide sequence that is complementary to another endogenous gene (target sequence).

Methods for antisense suppression can be used to reduce or eliminate expression of the targeted gene. The methods of antisense suppression comprise transforming a plant cell with at least one plant transcription unit comprising a promoter that drives expression in the plant cell operably linked to at least one nucleotide sequence that is antisense to a nucleotide sequence transcript of the target gene. By “antisense suppression” is intended the use of nucleotide sequences that are antisense to nucleotide sequence transcripts of endogenous plant genes to suppress the expression of those genes in the plant.

Methods for suppressing gene expression in plants using nucleotide sequences in the antisense orientation are known in the art. Antisense nucleotides are constructed to hybridize with the corresponding mRNA. Modifications to the antisense sequences may be made as long as the sequences hybridize to, and interfere with, expression of the corresponding mRNA. In this manner, antisense constructions having at least about 70%, 75%, 80%, 85%, 90%, 91%, 92%, 93%, 94%, 95%, 96%, 97%, 98%, or 99% or more sequence identity to the corresponding antisense sequences may be used. Furthermore, portions, rather than the entire nucleotide sequence, of the antisense nucleotides may be used to disrupt the expression of the target gene. Generally, sequences of at least 10 nucleotides, 50 nucleotides, 100 nucleotides, 200 nucleotides, or greater may be used.

The invention provides for the nucleic acid molecules above to be operably linked to a promoter. As described herein, the promoter may express constitutively or in a tissue-preferred manner. In one embodiment the promoter is an embryo-preferred promoter, and in another embodiment, an endosperm-preferred promoter. By “promoter” is intended a regulatory region of DNA usually comprising a TATA box capable of directing RNA polymerase II to initiate RNA synthesis at the appropriate transcription initiation site for a particular coding sequence. A promoter can additionally comprise other recognition sequences generally positioned upstream or 5′ to the TATA box, referred to as upstream promoter elements, which influence the transcription initiation rate.

Promoters that drive expression in a plant cell can be employed in the invention. Examples of promoters that can be used include, but are not limited to, the constitutive viral promoters such as the cauliflower mosaic virus (CaMV) 19S and 35S promoters or the figwort mosaic virus 35S promoter. See Kay et al., (1987) Science 236:1299 and European patent application No. 0 342 926; and the ubiquitin promoter (see for example U.S. Pat. No. 5,510,474) or any other ubiquitin-like promoter, which encodes a ubiquitin protein, but may have varying particular sequences (for example U.S. Pat. Nos. 5,614,399 and 6,054,574) the promoter for the small subunit of ribulose-1,5-bis-phosphate carboxylase, or promoters from the tumor-inducing plasmids from Agrobacterium tumefaciens, such as the nopaline synthase and octopine synthase promoters; the barley lipid transfer protein promoter, LTP2 (Kalla et al., Plant J. (1994) 6(6): 849-60); the END2 promoter (Linnestad et al. U.S. Pat. No. 6,903,205); and the polygalacturonase PG47 promoter (See Allen and Lonsdale, Plant J. (1993) 3:261-271; WO 94/01572; U.S. Pat. No. 5,412,085. See international application WO 91/19806 for examples of illustrative plant promoters.

Tissue-preferred promoters can be utilized to target enhanced transcription and/or expression within a particular plant tissue. Such promoters may express in the tissue of interest, along with expression in other plant tissue, may express strongly in the tissue of interest and to a much lesser degree than other tissue, or may express highly preferably in the tissue of interest. Tissue-preferred promoters include, but are not limited to, those described in Yamamoto et al. (1997) Plant J. 12(2): 255-265; Kawamata et al. (1997) Plant Cell Physiol. 38(7): 792-803; Hansen et al. (1997) Mol. Gen. Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Matsuoka et al. (1993) Proc Natl. Acad. Sci. USA 90(20): 9586-9590; and Guevara-Garcia et al. (1993) Plant J. 4(3): 495-505.

When expressing the ODP1 or the ZmDGAT-1 nucleic acid molecule, or FAD2-1 or FAD2-2 interfering fragments, the promoter may be any promoter, including a constitutive promoter, provided that it expresses strongly in the embryo tissue of the seed. In one embodiment, the promoter is one which drives expression preferentially in embryo tissue. In another embodiment, the embryo-preferred promoter is one which expresses by at least ten days after pollination and in another embodiment, expresses prior to 20 days after pollination. Examples of promoters which fall into these categories are the EAP1 promoter (Abbitt et al, U.S. Pat. Nos. 7,081,566 and 7,321,031); oleosin promoters (See, Plant et al Plant Mol Biol 25(2) 193-205 (1994) and, for example, the 16 kDa SB oleosin gene promoter of Glassman et al., U.S. Patent application 20090038034; the jasmonate-induce promoter (Jip1) (Abbitt et al., U.S. Pat. No. 7,432,418), and myo-inosital 1 phosphate synthase promoter (mi1ps3) as described in Abbitt et al, U.S. Pat. No. 7,432,418.

When expressing the 27 kDa interfering fragments, a constitutive promoter may be employed, provided that it expresses strongly in the endosperm tissue of the seed. In one embodiment, an endosperm-preferred promoter is employed. Examples include the 19 kDa alpha-zein promoter of cZ19B1 (See Lappegard et al, U.S. Pat. No. 6,225,529) and the Legumin 1 promoter (Abbitt et al, U.S. Pat. No. 7,211,712). The foregoing are provided by way of exemplification and are not intended to limit the scope of promoters that may be employed in the invention, provided the promoter expresses strongly in either the embryo tissue or endosperm tissue, as noted.

The promoter can be modified to provide for a range of expression levels of the heterologous nucleotide sequence. Less than the entire promoter region can be utilized and the ability to drive expression retained. However, it is recognized that expression levels of mRNA can be decreased with deletions of portions of the promoter sequence. Thus, the promoter can be modified to be a weak or strong promoter. Generally, by “weak promoter” is intended a promoter that drives expression of a coding sequence at a low level. By “low level” is intended levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts. Generally, at least about 30 nucleotides of an isolated promoter sequence will be used to drive expression of a nucleotide sequence. It is recognized that to increase transcription levels, enhancers can be utilized in combination with the promoter regions of the invention. Enhancers are nucleotide sequences that act to increase the expression of a promoter region. Enhancers are known in the art and include the SV40 enhancer region, the 35S enhancer element, and the like.

The range of available plant compatible promoters includes inducible promoters. An inducible regulatory element is one that is capable of directly or indirectly activating transcription of one or more DNA sequences or genes in response to an inducer. In the absence of an inducer the DNA sequences or genes will not be transcribed. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory element may be exposed to an inducer by externally applying the inducer to the cell or plant such as by spraying, watering, heating or similar methods. Any inducible promoter can be used in the instant invention. See Ward et al. Plant Mol. Biol. 22: 361-366 (1993). Exemplary inducible promoters include ecdysone receptor promoters, U.S. Pat. No. 6,504,082; promoters from the ACE1 system which responds to copper (Mett et al. PNAS 90: 4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991) and Gatz et al., Mol. Gen. Genetics 243: 32-38 (1994)); the maize GST promoter, which is activated by hydrophobic electrophilic compounds that are used as pre-emergent herbicides; and the tobacco PR-1a promoter, which is activated by salicylic acid. Other chemical-regulated promoters examples include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

In one embodiment, a construct of the invention comprises: a plant transcription unit comprising a maize ODP1 coding sequence operably linked to a promoter which expresses in the embryo; an RNAi plant transcription unit comprising a promoter which expresses in the embryo operably linked to two complementary nucleic acid fragments of at least 20 base pairs of the maize FAD2-1 coding region fused to two complementary fragments of at least 20 base pairs of the maize AGP2 coding region; and an RNAi plant transcription unit comprising a promoter that expresses in the endosperm operably linked to two complementary nucleic acid fragments of at least 20 base pairs of a maize 27 kDA gamma zein coding region. In all plant transcription units, transcription is stopped by a terminator derived from a plant gene or the synthetic terminator ALLSTOPS (see U.S. Pat. Pub. 2009/0038034).

In another embodiment, a construct of the invention comprises: a plant transcription unit comprising a variant of a maize DGAT1-2 coding region operably linked to a promoter that expresses in the embryo; an RNAi plant transcription unit comprising a promoter that expresses in the embryo, operably linked to two complementary nucleic acid fragments of at least 20base pairs of the maize FAD2-2 coding region; and an RNAi plant transcription unit comprising a promoter that expresses in the endosperm, operably linked to two complementary nucleic acid fragments of at least 20 base pairs of a 27 kDA gamma zein gene. In all plant transcription units, transcription is stopped by a terminator derived from a plant gene or the synthetic terminator ALLSTOPS (supra).

The maize Ovule Development Protein 1 nucleic acid molecule, also known as ODP1, refers to a CKC-like Aintegumenta transcription factor containing an AP2 domain and is described at Allen et al., U.S. Pat. No. 7,157,621. The sequence has been demonstrated to contribute to increased oil content in the seed.

Sequences which interfere, disrupt or otherwise down-regulate or limit expression of a fatty acid desaturase gene in the plant are also provided in the constructs. These delta-12 desaturases catalyze formation of a double bond between carbon positions 6 and 7 (numbered from the methyl end) (i.e., those that correspond to carbon positions 12 and 13 (numbered from the carbonyl carbon) of an 18 carbon-long fatty acyl chain.

The FAD2-1 gene is described at Lightner et al., U.S. Pat. No. 6,372,965 and also at WO94/11516. The FAD2-2 coding region is a truncation of the FAD2-1 gene, described at Shen et al., U.S. Pat. No. 7,008,664. The invention in an embodiment provides fragments of the FAD2-1 or FAD2-2 nucleotide sequence of at least 100 base pairs in a construct where the fragments are complementary. When referring to the FAD2-1 or FAD2-2 gene it is intended to refer to the nucleotide sequences as described above and which encode the amino acid described above and in the '965 patent and '644 patents.

Sequences which interfere with expression of a maize 27 kDa gamma zein gene are further provided. It has been demonstrated that down-regulation of 27 kDa gamma zein increases digestibility of the resulting grain.

The gene employed in an embodiment of the invention is disclosed in GenBank Accession No. AF371261 and GenBank Accession No. P04706. The invention in an embodiment is directed to using fragments of the 27 kDa gamma zein, in sense and complementary orientation.

A mutation of a type-1 diacylgycerol O-acyltransferase (DGAT) known as ZmDGAT1-2 has also been demonstrated to contribute to high oil, and to high oleic acid content, and is described at Allen et al., U.S. Patent Application No. 20070266462. This mutation differs from wild-type maize DGAT1-2 (sequence 52 in the '462 application) by having a glycine residue substitution for valine at the amino acid position 45 of the wild-type maize sequence, a serine residue substitution for the proline residue position 55, a deletion of the glutamine residue at positions 64, 65, 66, or 67; and/or an insertion of a phenylalanine at position located between: (a) the tryptophan residue at amino acid position 467 and the phenylalanine residue at amino acid position 468, (b) the phenylalanine residue at amino acid position 468 and the phenylalanine residue at amino acid position 469, and (c) the phenylalanine residue at amino acid position 469 and the serine residue at amino acid position 470. The high oil/high oleic acid DGAT1-2 of the invention can have one or more of these various alterations. When referring to the nucleic acid molecule of DGAT1-2, it is intended to refer to the mutated DGAT1-2 as described above and in the '462 application.

As used herein, “variants” of polynucleotides or polypeptides, are polynucleotides or polypeptides that differ from a reference polynucleotide or polypeptide, respectively. Generally, differences are limited such that the nucleotide sequences of the reference and the variant are closely similar overall and, in many regions, identical. Changes in the nucleotide sequence of the variant may be silent, that is, they may not alter the amino acids encoded by the polynucleotide. Where alterations are limited to silent changes of this type, a variant will encode a polypeptide with the same amino acid sequence as the reference. Additionally, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence. A variant and reference polypeptide may differ in amino acid sequence by one or more substitutions, additions, deletions, fusions and truncations, which may be present in any combination. It is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule.

The production of an appropriate expression construct will depend upon the host and the method of introducing the construct into the host and such methods are well known to one skilled in the art. For eukaryotes, the construct can include regions that control initiation of transcription, as described above, and control processing.

Other components of the construct may be included, also depending upon intended use of the gene. Examples include selectable markers, targeting or regulatory sequences, stabilizing or leader sequences, introns etc. General descriptions and examples of plant expression constructs and reporter genes can be found in Gruber, et al., “Vectors for Plant Transformation” in Method in Plant Molecular Biology and Biotechnology, Glick et al eds; CRC Press pp. 89-119 (1993).

The plant transcription units can include at the 3′ terminus of the heterologous nucleotide sequence of interest, a transcriptional and translational termination region functional in plants. The termination region can be native with the promoter nucleotide sequence of the transcription unit, can be native with the DNA sequence of interest, or can be derived from another source. Convenient termination regions are available from the Ti-plasmid of A. tumefaciens, such as the octopine synthase and nopaline synthase termination regions. See also, Guerineau et al. Mol. Gen. Genet. 262:141-144 (1991); Proudfoot, Cell 64:671-674 (1991); Sanfacon et al. Genes Dev. 5:141-149 (1991); Mogen et al. Plant Cell 2:1261-1272 (1990); Munroe et al. Gene 91:151-158 (1990); Ballas et al. Nucleic Acids Res. 17:7891-7903 (1989); Joshi et al. Nucleic Acid Res. 15:9627-9639 (1987).

The plant transcription units can additionally contain 5′ leader sequences. Such leader sequences can act to enhance translation. Translation leaders are known in the art and include by way of example, picornavirus leaders, EMCV leader (Encephalomyocarditis 5′ noncoding region), Elroy-Stein et al. Proc. Nat. Acad. Sci. USA 86:6126-6130 (1989); potyvirus leaders, for example, TEV leader (Tobacco Etch Virus), Allison et al.; MDMV leader (Maize Dwarf Mosaic Virus), Virology 154:9-20 (1986); human immunoglobulin heavy-chain binding protein (BiP), Macejak et al. Nature 353:90-94 (1991); untranslated leader from the coat protein mRNA of alfalfa mosaic virus (AMV RNA 4), Jobling et al. Nature 325:622-625 (1987); Tobacco mosaic virus leader (TMV), Gallie et al. (1989) Molecular Biology of RNA, pages 237-256; and maize chlorotic mottle virus leader (MCMV) Lommel et al. Virology 81:382-385 (1991). See also Della-Cioppa et al. Plant Physiology 84:965-968 (1987). The transcription unit can also contain sequences that enhance translation and/or mRNA stability such as introns.

In those instances where it is desirable to have the expressed product of the heterologous nucleotide sequence directed to a particular organelle, particularly the plastid, amyloplast, or to the endoplasmic reticulum, or secreted at the cell's surface or extracellularly, the plant transcription unit can further comprise a coding sequence for a transit peptide. Such transit peptides are well known in the art and include, but are not limited to, the transit peptide for the acyl carrier protein, the small subunit of RUBISCO, plant EPSP synthase, Zea mays Brittle-1 chloroplast transit peptide (Nelson et al. Plant physiol 117(4):1235-1252 (1998); Sullivan et al. Plant Cell 3(12):1337-48; Sullivan et al., Planta (1995) 196(3):477-84; Sullivan et al., J. Biol. Chem. (1992) 267(26):18999-9004) and the like. One skilled in the art will readily appreciate the many options available in expressing a product to a particular organelle. For example, the barley alpha amylase sequence is often used to direct expression to the endoplasmic reticulum (Rogers, J. Biol. Chem. 260:3731-3738 (1985)). Use of transit peptides is well known (e.g., see U.S. Pat. Nos. 5,717,084; 5,728,925).

In preparing the expression cassette or plant transcription unit, the various DNA fragments can be manipulated, so as to provide for the DNA sequences in the proper orientation and, as appropriate, in the proper reading frame. Toward this end, adapters or linkers can be employed to join the DNA fragments or other manipulations can be involved to provide for convenient restriction sites, removal of superfluous DNA, removal of restriction sites, or the like. For this purpose, in vitro mutagenesis, primer repair, restriction digests, annealing, and re-substitutions, such as transitions and transversions, can be involved.

As noted herein, the present invention provides vectors capable of expressing genes of interest. In general, the vectors should be functional in plant cells. At times, it may be preferable to have vectors that are functional in E. coli (e.g., production of protein for raising antibodies, DNA sequence analysis, construction of inserts, obtaining quantities of nucleic acids). Vectors and procedures for cloning and expression in E. coli are discussed in Sambrook et al. (supra).

Reporter genes can be included in the transformation vectors. Examples of suitable reporter genes known in the art can be found in, for example, Jefferson et al. (1991) in Plant Molecular Biology Manual, ed. Gelvin et al. (Kluwer Academic Publishers), pp. 1-33; DeWet et al. Mol. Cell. Biol. 7:725-737 (1987); Goff et al. EMBO J. 9:2517-2522 (1990); Kain et al. BioTechniques 19:650-655 (1995); and Chiu et al. Current Biology 6:325-330 (1996).

Selectable reporter genes for selection of transformed cells or tissues can be included in the transformation vectors. These can include genes that confer antibiotic resistance or resistance to herbicides. Examples of suitable selectable marker genes include, but are not limited to, genes encoding resistance to chloramphenicol, Herrera Estrella et al. EMBO J. 2:987-992 (1983); methotrexate, Herrera Estrella et al. Nature 303:209-213 (1983); Meijer et al. Plant Mol. Biol. 16:807-820 (1991); hygromycin, Waldron et al. Plant Mol. Biol. 5:103-108 (1985), Zhijian et al. Plant Science 108:219-227 (1995); streptomycin, Jones et al. Mol. Gen. Genet. 210:86-91 (1987); spectinomycin, Bretagne-Sagnard et al. Transgenic Res. 5:131-137 (1996); bleomycin, Hille et al. Plant Mol. Biol. 7:171-176 (1990); sulfonamide, Guerineau et al. Plant Mol. Biol. 15:127-136 (1990); bromoxynil, Stalker et al. Science 242:419-423 (1988); glyphosate, Shaw et al. Science 233:478-481 (1986); and phosphinothricin, DeBlock et al. EMBO J. 6:2513-2518 (1987).

Scorable or screenable markers may also be employed, where presence of the sequence produces a measurable product. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al. The EMBO Journal vol. 6 No. 13 pp. 3901-3907); and alkaline phosphatase. Other screenable markers include the anthocyanin/flavonoid genes in general (See discussion at Taylor and Briggs, The Plant Cell (1990) 2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., Plant Cell (1996) 8: 1171-1179; Scheffler et al. Mol. Gen. Genet. (1994) 242:40-48) and maize C2 (Wienand et al., Mol. Gen. Genet. (1986) 203:202-207); the B gene (Chandler et al., Plant Cell (1989) 1:1175-1183), the p1 gene (Grotewold et al, Proc. Natl. Acad. Sci. USA (1991) 88:4587-4591; Grotewold et al., Cell (1994) 76:543-553; Sidorenko et al., Plant Mol. Biol. (1999) 39:11-19); the bronze locus genes (Ralston et al., Genetics (1988) 119:185-197; Nash et al., Plant Cell (1990) 2(11): 1039-1049), among others. Yet further examples of suitable markers include the cyan fluorescent protein (CYP) gene (Bolte et al. (2004) J. Cell Science 117: 943-54 and Kato et al. (2002) Plant Physiol 129: 913-42), the yellow fluorescent protein gene (PhiYFP™ from Evrogen; see Bolte et al. (2004) J. Cell Science 117: 943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989) EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al. (2002) Biotechniques 2(2):286-293). Additional examples include a p-lactamase gene (Sutcliffe, Proc. Nat'l. Acad. Sci. U.S.A. (1978) 75:3737), which encodes an enzyme for which various chromogenic substrates are known (e.g., PADAC, a chromogenic cephalosporin); a xy1E gene (Zukowsky et al., Proc. Nat'l. Acad. Sci. U.S.A. (1983) 80:1101), which encodes a catechol dioxygenase that can convert chromogenic catechols; an α-amylase gene (Ikuta et al., Biotech. (1990) 8:241); and a tyrosinase gene (Katz et al., J. Gen. Microbiol. (1983) 129:2703), which encodes an enzyme capable of oxidizing tyrosine to DOPA and dopaquinone, which in turn condenses to form the easily detectable compound melanin. Clearly, many such markers are available to one skilled in the art.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription or transcript and translation of the sequence to effect phenotypic changes in the organism. Thus, any method which provides for efficient transformation/transfection may be employed.

Methods for introducing expression constructs into plant tissue available to one skilled in the art are varied and will depend on the plant selected. Procedures for transforming a wide variety of plant species are well known and described throughout the literature. See, for example, Miki et al, “Procedures for Introducing Foreign DNA into Plants” in Methods in Plant Molecular Biotechnology, supra; Klein et al, Bio/Technology 10:268 (1992); and Weising et al., Ann. Rev. Genet. 22: 421-477 (1988). For example, the DNA construct may be introduced into the genomic DNA of the plant cell using techniques such as microprojectile-mediated delivery, Klein et al., Nature 327: 70-73 (1987); electroporation, Fromm et al., Proc. Natl. Acad. Sci. 82: 5824 (1985); polyethylene glycol (PEG) precipitation, Paszkowski et al., EMBO J. 3: 2717-2722 (1984); direct gene transfer WO 85/01856 and EP No. 0 275 069; in vitro protoplast transformation, U.S. Pat. No. 4,684,611; and microinjection of plant cell protoplasts or embryogenic callus, Crossway, Mol. Gen. Genetics 202:179-185 (1985). Co-cultivation of plant tissue with Agrobacterium tumefaciens is another option, where the DNA constructs are placed into a binary vector system. See e.g., U.S. Pat. No. 5,591,616; Ishida et al., “High Efficiency Transformation of Maize (Zea mays L.) mediated by Agrobacterium tumefaciens” Nature Biotechnology 14:745-750 (1996). The virulence functions of the Agrobacterium tumefaciens host will direct the insertion of the construct into the plant cell DNA when the cell is infected by the bacteria. See, for example Horsch et al., Science 233: 496-498 (1984), and Fraley et al., Proc. Natl. Acad. Sci. 80: 4803 (1983).

Following transformation, regeneration is needed to obtain a whole plant from transformed cells. Techniques for regenerating plants from tissue culture such as transformed protoplasts or callus cell lines, are known in the art. For example, see Phillips, et al.; Plant Cell Tissue Organ Culture; Vol. 1: p 123; (1981); Patterson, et al.; Plant Sci.; Vol. 42; p. 125; (1985); Wright, et al.; Plant Cell Reports; Vol. 6: p. 83; (1987); and Barwale, et al.; Planta; Vol. 167; p. 473 (1986); each incorporated herein in its entirety by reference. The selection of an appropriate method is within the skill of the art.

Regeneration of transgenic plant tissue is dependent on the transformation protocol used. Generally, embryogenic tissue is subcultured to a medium comprising appropriate amounts of components including, but not limited to: salts, vitamins, plant hormones and antibiotics. The tissue is then incubated until the development of well-formed, matured somatic embryos can be seen. The embryos are individually subcultured to a germination medium and incubated until the somatic embryos have germinated and produced a well-defined shoot and root. The individual plants are subcultured to germination medium to allow further plant development. When the plants are well-established, they are transplanted to horticultural soil, hardened off, and potted into commercial greenhouse soil mixture and grown to sexual maturity in a greenhouse. An elite inbred line can be used as a male to pollinate regenerated transgenic plants.

The foregoing methods for transformation would typically be used for producing transgenic inbred lines. Transgenic inbred lines could then be crossed, with another (non-transformed or transformed) inbred line, in order to produce a transgenic hybrid maize plant. Alternatively, a genetic trait which has been engineered into a particular maize line using the foregoing transformation techniques could be moved into another line using traditional backcrossing techniques that are well known in the plant breeding arts.

Plants are selected for advancement using any of several methods known to those of skill in the art. Cells or tissues carrying polynucleotides or polypeptides may be detected at the DNA level by a variety of techniques well-known to those of skill in the art. Such assay methods include radioimmunoassays, competitive-binding assays, Western Blot analysis and ELISA assays. The use of polynucleotides in markers to assist in breeding programs, is described for example in PCT publication US89/00709. The polynucleotides may be used directly for detection or may be amplified enzymatically by using PCR prior to analysis. PCR (Saiki et al., Nature 324:163-166 (1986)). Detection of a specific DNA sequence may be achieved by methods such as hybridization, RNase protection, chemical cleavage, direct DNA sequencing or the use of restriction enzymes, (e.g., restriction fragment length polymorphisms (“RFLP”) and Southern blotting of genomic DNA. Plants can also be selected for advancement by visual observation or analysis of phenotypic characteristics such as the assays described herein.

A backcrossing approach can be used to move an engineered trait from a public, non-elite line into an elite line, or from a hybrid maize plant containing a foreign gene in its genome into a line or lines which do not contain that gene. As used herein, “crossing” can refer to a simple X by Y cross, or the process of backcrossing, depending on the context. The plants of the invention may also be used in the TopCross® method of breeding in which a blend of two types of corn are planted, one is a hybrid that is the grain parent female, the other is the pollinator. The pollinator is enhanced in a quality grain trait, is nonisogenic to the female plant and enhanced in the grain trait. The result is a harvest of high yield corn grain enhanced in the quality grain trait. See Bergquist et al, U.S. Pat. No. 5,706,603. The resulting blend progeny will be 75% transgenic and 25% wild-type. The introduced constructs of the invention are capable of sorting on a single locus in one embodiment of the invention wherein they transfer and are inherited as a unit, providing consistent phenotypic expression when introduced into another plant.

In certain embodiments the polynucleotides of the present invention can be stacked with any combination of polynucleotide sequences of interest in order to create plants with a desired trait. A trait, as used herein, refers to the phenotype derived from a particular sequence or groups of sequences. For example, one or more of the polynucleotides of the present invention, which confer the increased oil, oleic acid content and increased digestibility phenotype, may be stacked with any other polynucleotides encoding polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like.

The combinations generated can also include multiple copies of any one of the polynucleotides of interest. The polynucleotides of the present invention can also be stacked with any other gene or combination of genes to produce plants with a variety of desired trait combinations including, but not limited to, balanced amino acids (e.g., hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122) and high methionine proteins (Pedersen et al. (1986) J. Biol. Chem. 261:6279; Kirihara et al. (1988) Gene 71:359 and Musumura et al. (1989) Plant Mol. Biol. 12:123); and thioredoxins (Sewalt et al., U.S. Pat. No. 7,009,087).

The polynucleotides of the present invention can also be stacked with traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)). One could also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821).

These stacked combinations can be created by any method including, but not limited to, cross-breeding plants by any conventional or TopCross® methodology, or genetic modification. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of another polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention. All vectors were constructed using standard molecular biology techniques (Sambrook et al. (1989) Molecular Cloning: A Laboratory Manual (2d ed., Cold Spring Harbor Laboratory Press, Plainview, N.Y.).

### EXAMPLES

### Example 1

### Construction of PHP27347

Four plant transcription units (PTU) were inserted between the T-DNA borders of a superbinary vector pSB11 obtained from Japan Tobacco Inc. (Tokyo, Japan, disclosed in U.S. Pat. No. 5,591,616).

The first PTU comprised a complement of a fragment of the maize 27 kD gamma-zein coding region (GenBank Accession No: AF371261) fused to a complement of the ADH1 intron 1 (Dennis et al., Nucl. Acids Res. 12:3983-3990, 1984), fused to the same gamma-zein fragment in sense orientation and operably linked to the CZ19B1 promoter (U.S. Pat. No. 6,225,529) which expresses in endosperm. The polynucleotide sequence of this PTU is from position 15 to position 2250 of SEQ ID NO:1.

The second PTU was generated by fusing a truncation of the maize AGP2 small subunit coding region in sense orientation (GenBank Accession No: AYO32604, published Apr. 30, 2001) to two fragments of the maize FAD2 coding region (U.S. Pat. No. 6,372,965) in complementary orientation followed by a fragment of the maize ADH1 intron 1 in complementary orientation, followed by the FAD2 fragments in sense orientation, the AGP2 fragment in complementary orientation, and operably linked to a complement of the maize 16 kD oleosin promoter (positions 4674-5632 of SEQ ID NO:1) which expresses in the embryo. This PTU sequence is from position 2488 to position 5632 of SEQ ID NO:1.

The third PTU was generated by operably linking the maize EAP1 promoter (Abbitt et al, U.S. Pat. Nos. 7,081,566 and 7,321,031) which expresses in embryo, to the maize ODP1 coding region (Allen, et al, U.S. Pat. No. 7,157,621). This PTU is from positions 5788-9024 of SEQ ID NO:1.

All PTUs included either a plant-derived terminator or the synthetic ALLSTOPS element (US Pat Pub: 2009/0038034) which is a series of stop codons designed to stop all six open reading frames. Recombination sites were also inserted between PTUs.

The selection cassette comprised the maize ubiquitin promoter, 5′ UTR (Christensen et al. (1989) Plant Mol. Biol. 12:619-632 and Christensen et al. (1992) Plant Mol. Biol. 18:675-689) and intron (GenBank Accession No. 594464) fused to the MO-PAT coding region (Wohlleben et al. (1988) Gene 70:25-37). Recombination sites were inserted between PTUs.

A selectable marker cassette comprising genes for spectinomycin and tetracycline resistance was inserted proximal to the left T-DNA border.

### Example 2

### Construction of PHP30935

Four plant transcription units (PTU) were inserted between the T-DNA borders of a superbinary vector pSB11 obtained from Japan Tobacco Inc (supra).

The first PTU comprised a fragment of the complement of the maize FAD2 coding region fused to a variant of the fragment in sense orientation further fused to a complement of the maize 16 kD oleosin promoter. This PTU is from positions 14-2586 of SEQ ID NO:2. A recombination site and the ALLSTOPS element were inserted proximal to the PTU.

The second PTU was generated by fusing a complement of a fragment of the maize 27 kD gamma-zein coding region and the maize ADH1 intron 1 to the same gamma-zein fragment in sense orientation. These elements were further operably linked to a complement of the CZ19B1 promoter and the ALLSTOPS element. This PTU is from positions 2783-5014 of SEQ ID NO:2.

The third PTU comprised the maize 16 kD oleosin promoter fused to a variant of the maize DGAT1-2 coding region (Allen et al., US Patent Application No. 20070266462) operably linked to the NOS terminator (nopaline synthase derived from Agrobacterium). This PTU is from positions 5226-8035 of SEQ ID NO:2.

The selection cassette comprised the maize ubiquitin promoter, 5′ UTR (Christensen et al. (1989) Plant Mol. Biol. 12:619-632 and Christensen et al. (1992) Plant Mol. Biol. 18:675-689) and intron (GenBank Accession No. S94464) fused to the MO-PAT coding region (Wohlleben et al. (1988) Gene 70:25-37). Recombination sites were inserted between PTUs.

### Example 3

### Transformation of Maize Cells

For Agrobacterium-mediated transformation of maize, the constructs described above were prepared, and the method of Zhao was employed (U.S. Pat. No. 5,981,840, and PCT patent publication WO98/32326).

Briefly, immature embryos were isolated from maize and the embryos contacted with a suspension of Agrobacterium, where the bacteria are capable of transferring the nucleotide sequence of interest to at least one cell of at least one of the immature embryos (step 1: the infection step). In this step the immature embryos were immersed in an Agrobacterium suspension for the initiation of inoculation. The embryos were co-cultured for a time with the Agrobacterium (step 2: the co-cultivation step). The immature embryos were cultured on solid medium following the infection step. Following this co-cultivation period an optional “resting” step is contemplated. In this resting step, the embryos were incubated in the presence of at least one antibiotic known to inhibit the growth of Agrobacterium without the addition of a selective agent for plant transformants (step 3: resting step). The immature embryos were cultured on solid medium with antibiotic, but without a selecting agent, for elimination of Agrobacterium and for a resting phase for the infected cells. Next, inoculated embryos were cultured on medium containing a selective agent and growing transformed callus was recovered (step 4: the selection step). The immature embryos were cultured on solid medium with a selective agent resulting in the selective growth of transformed cells. The callus was then regenerated into plants (step 5: the regeneration step), and calli grown on selective medium were cultured on solid medium to regenerate the plants.

The regenerated plants were advanced genetically, predominately via backcrossing strategies, to create material suitable for analysis, energy availability studies, and product development applications.

Grain from progeny of these plants were tested in the in vitro digestibility assay (EDDM), for oil content, and for oleic acid content as described herein.

### Example 4

### Oil Measurement

Kernels from each mature ear were analyzed by NMR to determine the amount of oil as well as the concentration of oil on a per kernel basis. For seed oil, air-dried kernels were used for direct NMR measurements.

Results from analysis of grain from 10 hybrids backcrossed with the '347 and '935 constructs is shown in Tables 1 and 2.

### Example 5

### Oleic Acid Measurement

After determination of total oil content, fatty acids were determined following a transmethylation step. The resulting methyl esters of the fatty acids were separated, and their concentrations determined by use of capillary gas chromatography in accordance with standard operating procedures known in the art (see Moon et al. (2000) Lipids 35:471-479).

Results from analysis of grain from 10 hybrids backcrossed with the '347 and '935 constructs is shown in Tables 1 and 2.

### Example 6

### Digestibility Assay

### In Vitro Enzyme Digestible Dry Matter (EDDM) Assay:

Corn grain was ground in a micro Wiley Mill (Thomas Scientific, Swedesboro, N.J.) through a 1 mm screen; 0.5 g of ground corn sample was placed in a pre-weighed nylon bag (50 micron pore size) and heat sealed. Approximately 40 bags were placed in an incubation bottle with 2 L of 0.2M phosphate buffer (pH 2.0) containing pepsin (0.25 mg/ml). Samples were incubated in a Daisy II incubator (ANKOM Technology, Fairport, N.Y.) at 39° C. for 2 hours. After 2 hours, samples were placed in a mesh bag and washed for 2 minutes with cold water in a washer (Whirlpool) using delicate cycle. Samples were then transferred into 2 L of 0.2M phosphate buffer (pH 6.8) containing pancreatin (5.0 mg/ml) and incubated at 39° C. for 4 or 6 hours. Samples were washed for 2 minutes as described earlier. Samples were then dried overnight at 55° C. and weighed. The difference in sample weight before and after incubation was expressed as percentage of enzyme digestible dry matter digestibility (EDDM).

Results from analysis of grain from 10 hybrids backcrossed with the '347 and '935 constructs is shown in Tables 1 and 2.

TABLE 1
Compositional Data for PHP27347 in 10 Hybrids:

| | Null | Trans | % Diff. | ‘Std’ value |
|---|---|---|---|---|
| Oil (%) | 3.58a | 4.26b | 19 | 3.8 |
| Oleic (%) | 25.2 | 79.4 | 315 | 25-30 |
| Digestibility (%) | 86.8 | 88.86 | 2.4 | — |

TABLE 2
Compositional Data for PHP30935 in 10 Hybrids:

| | Null | Trans | % Diff. | ‘Std’ value |
|---|---|---|---|---|
| Oil (%) | 3.5 | 4.4 | 25.2 | 3.8 |
| Oleic (%) | 25.6 | 71.9 | 182 | 25-30 |
| Digestibility (%) | 87.7 | 90.2 | 2.9% | — |

Although the foregoing invention has been described in some detail by way of illustration and example for purposes of clarity of understanding, it will be apparent that certain changes and modifications can be practiced within the scope of the appended claims.

1. A genetically modified maize plant having grain with an oleic acid content of at least 60% on a dry weight basis, an oil content that is increased at least 15% on a dry weight basis, and digestibility that is increased at least 0.5% over an unmodified maize plant.

2. The plant of claim 1 wherein the oleic acid content is at least 70%.

3. The plant of claim 1 wherein the oil content is increased at least 20%.

4. The plant of claim 1 wherein digestibility is increased at least 1%.

5. The plant of claim 1 wherein the oleic acid content is at least 85%.

6. The plant of claim 1 wherein the oil content is increased at least 30%.

7. The plant of claim 1 wherein digestibility is increased at least 2%.

8. The plant of claim 1 wherein the grain attains the oleic acid content, oil content, and increased digestibility due to genetic modification by transformation with the nucleic acid construct of SEQ ID NO:1.

9. The plant of claim 1 wherein the grain attains the oleic acid content, oil content, and increased digestibility due to genetic modification by transformation with the nucleic acid construct of SEQ ID NO:2.

10. The grain of the plant of claim 1.

11. A method of producing a maize plant having grain with an oleic acid content of at least 60% on a dry weight basis, an oil content that is increased at least 15% on a dry weight basis, and digestibility that is increased at least 1% over an unmodified maize plant, the method comprising:
a) introducing into a plant cell an construct with means for increasing oleic acid content, oil content, and digestibility;
b) regenerating a genetically modified plant from the cell; and
c) selecting for a genetically modified plant with grain having an oleic acid content of at least 60% on a dry weight basis, an oil content that is increased at least 15% on a dry weight basis, and digestibility that is increased at least 1% over an unmodified maize plant.

12. The method of claim 11 wherein the oleic acid content is at least 70%.

13. The method of claim 11 wherein the oil content is increased at least 20%.

14. The method of claim 11 wherein digestibility is increased at least 2%.

15. The method of claim 11 wherein the oleic acid content is at least 85%.

16. The method of claim 11 wherein the oil content is increased at least 30%.

17. The method of claim 11 wherein the construct is SEQ ID NO:1.

18. The method of claim 11 wherein the construct is SEQ ID NO:2.

19. The grain of the method of claim 11.

20. An isolated nucleic acid molecule comprising a nucleotide sequence selected from the group consisting of:
(a) the nucleotide sequence set forth in SEQ ID NO:1, or SEQ ID NO: 2 and
(b) functional variants of (a).
